(19) United States
(12) Patent Application Publication
(10) Pub. No.: US 2016/0206859 A1
(43) Pub. Date: Jul. 21, 2016

(54) GUIDE WIRE HOLDER FOR RECEIVING AND HOLDING A MEDICAL GUIDE WIRE AND FOR ATTACHING SAME TO A MEDICAL DEVICE, IN PARTICULAR AN ENDOSCOPE

(71) Applicant: MEDI-GLOBE GMBH, Rohrdorf-Achenmuehle (DE)

(72) Inventor: Ingo EDEN, Muenchen (DE)

(73) Assignee: MEDI-GLOBE GMBH, Rohrdorf-Achenmuehle (DE)

(21) Appl. No.: 14/914,060

(22) PCT Filed: Aug. 29, 2014
(86) PCT No.: PCT/DE2014/000445

§ 371 Date: Feb. 24, 2016

(30) Foreign Application Priority Data
Aug. 30, 2013 (DE) 20 2013 007 740.9

(51) Int. Cl. A61M 25/09; A61B 1/00; A61B 1/018

(52) U.S. Cl. CPC A61M 25/09; A61B 1/018; A61B 1/0014; A61M 2025/09125; A61M 2205/0216

(57) ABSTRACT

A guide wire holder for receiving and holding an inherently elastic medical guide wire and for attaching same to a medical device, such as an endoscope, has an attachment part through which the guide wire can he guided and a guide wire support which is connected to the attachment part projects from same, and allows the guide wire to be held by a guide wire receiving device. The guide wire receiving device contains a first guide wire deflecting element in the form of a deflecting and clamping bracket which is open on one side, the guide wire deflecting element guiding the guide wire away from the attachment part and clamping the guide wire, arid the guide wire receiving device contains two additional guide wire deflecting elements. The deflecting and clamping bracket has two bracket parts which project from the guide wire support and between which a slot is provided. The slot runs between the two bracket parts in one position and/or alignment located outside of a guide wire expansion path of the guide wire support, the expansion path being determined by the inherent elasticity of the guide wire.

The invention relates to a guide wire holder for accommodation and holding in place of a medical guide wire that possesses inherent elasticity, and for attachment to a medical device, particularly to an endoscope, having an attachment part through which the guide wire can be passed, and a guide wire support connected with the attachment part and projecting away from the latter, which support permits the guide wire to be held in place by means of a guide wire accommodation device, which device contains a first guide wire deflection member that deflects and clamps the guide wire at a distance from the attachment part, in a direction leading away from its passage direction in the attachment, part, which member has a deflection and clamping bracket that is open on one side and rises from a flat initial region of the guide wire support, which bracket allows the guide wire to be accommodated in an opening and released from the latter again, and is followed, at further distances from the attachment part, by an additional guide wire deflection element and yet a further guide wire deflection element.

A guide wire holder of the type indicated above is already known (DE 10 2010 011 222 A1; WO 2011/110152 A1). In a concrete exemplary embodiment of this known guide wire holder, the deflection and clamping bracket that is open on one side is formed by a one-armed bracket that projects away from the flat initial region with a single support part and is open on the bracket side that lies opposite the support part. Fundamentally, however, the one-sided opening of the deflection or clamping bracket can also be provided at another location of the deflection or clamping bracket, such as, for example, on its top. In this case, the deflection and clamping bracket will then contain two bracket arms, between which the aforementioned opening is situated. In this regard, however, special provisions must be made in order to guarantee secure accommodation and holding in place of the guide wire under such a deflection and clamping bracket.

The present invention is therefore based on the task of showing a way how such secure accommodation and holding in place of the guide wire can be guaranteed under a two-armed deflection and clamping bracket, in the case of a guide wire holder of the type indicated initially.

The task indicated above is accomplished, according to the invention, in the case of a guide wire holder of the type stated initially, in that the deflection and clamping bracket has two bracket parts that project away from the initial region of the guide wire support, and between which a slot that forms the said opening is provided, which slot has a slot width that lies below the transverse dimension of the guide wire in the case of bracket parts that can be pressed elastically apart from one another, and a slot width that corresponds to the transverse dimension of the guide wire in the case of rigid bracket parts, and which is situated between the two bracket parts in a position and/or alignment outside of a guide wire expansion path of the guide wire support, which path is determined by the inherent elasticity of the guide wire.

The invention brings with it the advantage that secure accommodation and fixing in place of the guide wire under the deflection and clamping bracket is achieved by means of the fixation of the slot in a position and/or alignment outside of the guide wire expansion path away front the guide wire support, which path is determined by the inherent elasticity of the guide wire. As a result, the slot in question can be situated in a position within one of the two bracket parts, at a distance from the bracket center, and, in this connection, can run parallel to the longitudinal direction of the guide wire. This progression can even exist if the two bracket parts can be pressed apart from one another elastically, and the slot has a slot width that lies below the transverse dimension of the guide wire. In the event that the said slot is situated in a position in the region of the bracket center, however, it will preferably be aligned transverse to the longitudinal direction of the guide wire. This transverse alignment of the slot can be provided, however, when the latter is situated in a position outside of the region of the bracket center.

In connection with the guide wire mentioned, the following should still be noted here. Here, the aforementioned guide wire expansion path is understood to mean the path of the guide wire in the direction of which the guide wire, fixed in place at two fixation points, attempts to expand on the basis of its inherent elasticity. In the present case, the two fixation points of the guide wire exist on its progression path between its contact points or surfaces at the exit location from the cover plate of the attachment part and at the deflection and clamping bracket. On this progression path, the inherently elastic guide wire attempts to expand in a direction essentially perpendicular to this progression path, with the consequence that it presses against the underside of the deflection and clamping bracket or its bracket parts, which underside faces toward the guide wire support, on its expansion path. In general, the transverse dimension of the guide wire is understood to be its dimension that runs transverse, in other words at 90° relative to its longitudinal axis; in the case of a guide wire having a round cross-section, the respective dimension is determined by the diameter of the guide wire in question. If the guide wire used possesses a rectangular cross-section with different edge lengths, then its transverse dimension is understood to be the one edge length of this rectangular cross-section that has the shortest edge length. In the case of an oval cross-section having a large diameter and a small diameter, in comparison, the transverse dimension of the guide wire Is to be understood to be its small diameter.

According to a practical further development, of the invention, one of the two bracket parts has a support member that projects away from the initial region of the guide wire support, and an upper part carried by this member, having a cross-section or body volume that projects away relative to the cross-section of the support part, and the other bracket part is merely a support part adjacent to the said upper part, which support part is provided with one side at a distance of the slot width from the respective upper part of the one of the two bracket parts. In this way, the advantage is obtained that it is possible to make do with a relatively simple bracket design.

Preferably, the slot is provided only in the region of the projecting cross-section or body volume of the upper part of the one bracket part and the support part that forms the other bracket part, and the support member that carries this upper part is set back, if necessary, with its side that faces toward the slot, opposite the latter. In this way, an at times sufficient space for secure accommodation and holding in place of a guide wire introduced through the aforementioned slot can be made available underneath the said slot, in particularly simple manner—when looking at the guide wire holder from the side of the projecting cross-section or body volume of the upper part of the one bracket part.

According to another practical further development of the invention, each of the two bracket parts has a support member that projects away from the initial region of the guide wire support, and an upper part supported by the latter, having a cross-section or body volume that projects relative to the cross-section of the related support part, and the upper parts of the two bracket parts are provided at a distance from one another, forming the said slot. In this way, the advantage of a relatively simple bracket design is also obtained, which can furthermore be implemented with bracket parts that can be configured symmetrically, at least to a great extent.

Preferably, in the practical further development of the invention last considered, the support parts are set back, from edges of the slot of the upper parts supported by them, with their one sides that lie opposite one another. In this way, the advantage, is obtained that a relatively large space for secure accommodation and holding in place of a guide wire introduced through the said slot can be made available underneath the said slot—when looking at the guide wire holder from the side of the projecting cross-sections or body volumes of the upper parts of the two bracket parts.

According to yet another practical further development of the invention, the respective upper part, with its cross-section or body volume that projects relative to the cross-section of its support part, is configured in the shape of a roller, roll, cone, barrel or sphere. In this way, particularly effective bracket parts can be implemented for introduction into and holding in place of a guide wire in the bracket arrangement.

Preferably, the respective upper part forms a flattened surface toward the slot. This measure brings with it the advantage that the slot in question effectively and thereby better prevents the guide wire from slipping out of the space that is situated underneath the upper part of the respective having a cross-sections or body volumes that project relative to the cross-section of the related support part, or at least makes this more difficult than a slot structured in the form of a point or line. Furthermore, the flattened surface of the slot—when looking at the guide wire holder in the direction of the flattened surface—can possess an incline that deviates from the vertical.

It is practical if the slot has a straight-line progression. This brings with it the advantage that the slot in question can be produced in particularly simple manner.

Preferably, the slot provided in the central region of the deflection and clamping bracket, between its two bracket parts, runs at an angle of about 5° to about 175° with reference to the accomodation and holding in place path of the guide wire on the guide wire support. Within this angle range, the guide wire is particularly effectively prevented from slipping out of the space that is situated underneath the upper part of the respective having a cross-sections or body volumes that project relative to the cross-section of the related support part.

It is furthermore advantageous if the guide wire support has a material such as rubber or silicone in the region of the accommodation and holding in place path of the guide wire, which material exerts a friction resistance on the guide wire that lies against it. In this way, the guide wire is securely held in place, in particularly effective manner, in the space that is situated underneath the upper part of the respective having a cross-section or body volume that projects relative to the cross-section of the related support part.

The invention will be explained in greater detail below, using exemplary embodiments, using drawings.

The drawings show:

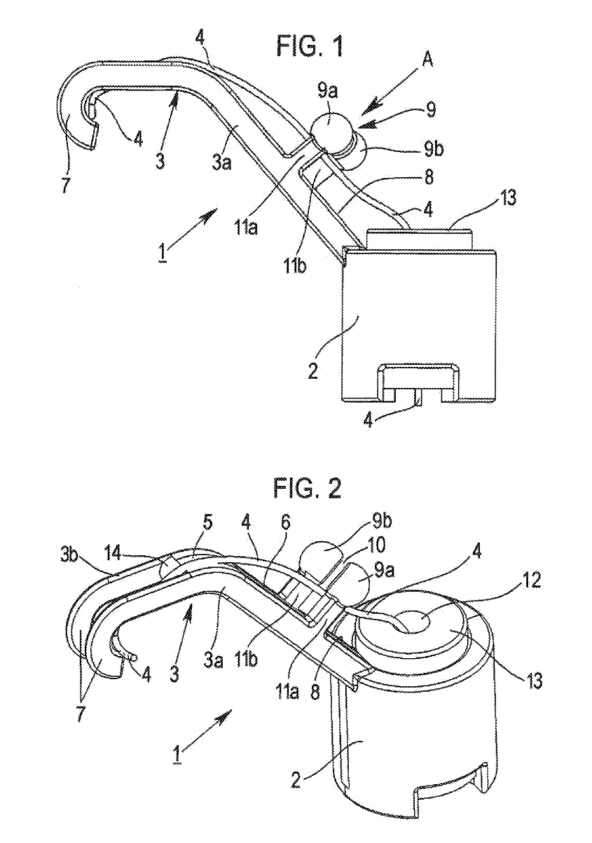

FIG. 1 a schematic side view of a guide wire holder according to an embodiment of the present invention, in a size that can be different from the size used in practice,

FIG. 2 a perspective view of the guide wire holder shown in FIG. 1, seen at a slant from above,

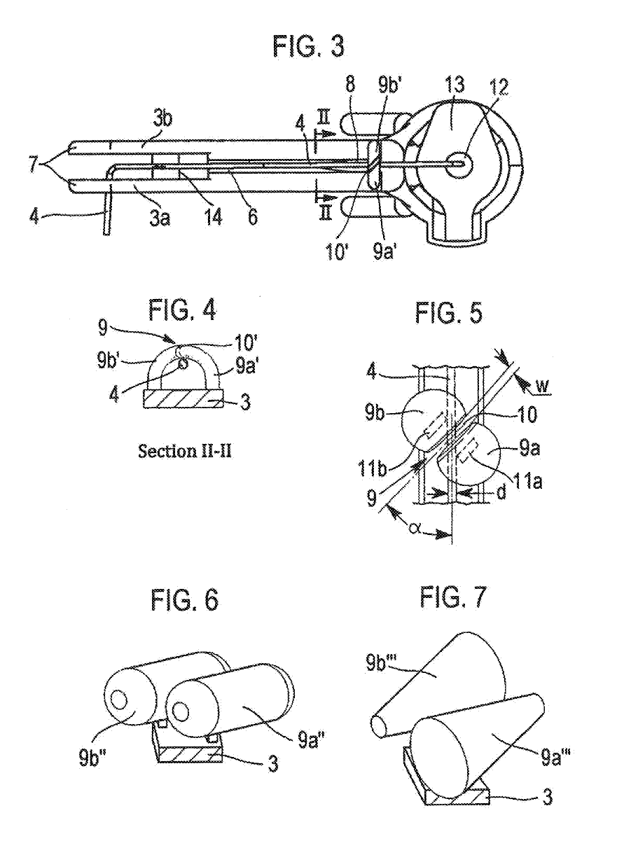

FIG. 3 a top view of the guide wire holder indicated in the document mentioned initially (DE 10 2010 011 222 A1; WO 2011/110152 A1), having a guide wire holder according to an embodiment of the present invention,

FIG. 4 a sectional view along the section plane II-II entered in FIG. 3,

FIG. 5 a top view, as a detail, of the guide wire holder shown in FIG. 1, in the viewing direction A indicated by an arrow there,

FIG. 6 a perspective view, as a detail, of a guide wire holder according to another embodiment of the invention, in a size that can be different from the size used in practice, and

FIG. 7 a perspective view, as a detail, of a guide wire holder according to yet another embodiment of the invention, in a size that can be different from the size used in practice.

Before the drawings are discussed in further detail, it should be noted that in all the drawings, devices or elements that are the same or correspond to one another are fundamentally labeled with the same reference symbols.

In FIG. 1, a guide wire holder 1 according to an embodiment of the present invention is shown in a side view. This guide wire holder 1, like the guide wire holder indicated in the document mentioned initially, contains an attachment part 2 for attachment of the guide wire holder 1 to a medical device (not shown here), which can particularly be an endoscope, to the working channel connector of which the attachment part 2 in question can be attached. Here, the attachment part 2 can also be a set-on part or a cap, for example, which can be set onto an accommodation part of the respective medical device and cannot be easily pulled off there. However, the attachment part 2 can also contain a screw connection part, if necessary, with which it can be screwed onto or into a corresponding screw connection part of the respective medical device.

A guide wire support 3 is connected with the attachment part 2, which support makes it possible to hold a medical guide wire 4 that, has inherent elasticity in place by means of a guide wire accommodation device. As is clearly evident from the perspective representation according to FIG. 2 and the top view according to 3, the guide wire support 3 contains two support arms 3a and 3b that are connected with one another, which are connected with one another by means of a connection part 6 that is preferably flat or runs in a straight line, in an initial region 8 between their connection location with the attachment part 2 and a termination location 5.

The guide wire support 3 and thereby the two support arms 3a and 3b of the latter have a progression that is directed upward at a slant to the left when looking at the side view according to FIG. 1. After a specific, predetermined height has been reached at the aforementioned termination location 5, the two support arms 3a and 3b run first approximately horizontally and then downward according to FIGS. 1 and 2, in other words bent away relative to the plane of the attachment part 2, and finally run out into a bent offset region 7 at their respective end.

According to FIGS. 1 and 2, the termination location 5 is formed by means of a support region that lies underneath the top of the guide wire support 3, which is shown in the upper part of these figures, in which region the guide wire 4 can be accommodated. The lowering of the guide path from the top of the guide wire support 3 lies between 0.5 mm and 4 mm, for example.

The guide wire accommodation device of the guide wire support 3 makes it possible to hold the guide wire 4 in place in such a manner that the latter cannot be displaced or pulled in its longitudinal direction, in its accommodated state. For this purpose, the guide wire accommodation device comprises, for one thing, a first guide wire deflection member 9 that rises from the flat initial region 8 of the guide wire support 3, starting from the attachment part 2, which member is configured as a deflection and clamping bracket 9 that is open on one side. This deflection and clamping bracket 9 consists of two bracket parts 9a and 9b, which project away from the initial region 8 of the guide wire support 3, and between which a slot 10 that forms the said one opening is provided, which slot here has a slot width w that corresponds to the transverse dimension d of the guide wire 4, and is situated here between the two bracket parts 9a, 9b, in a position and/or alignment outside of a guide wire expansion path of the guide wire support 3, which path is determined by the inherent elasticity of the guide wire 4. The slot width w that corresponds to the transverse dimension d of the guide wire 4 is equal to the transverse dimension d in question in the case of rigid bracket parts 9a, 9b; however, it can also be greater than this transverse dimension d.

As has already been mentioned above, here the guide wire expansion path mentioned is understood to be that path of the guide wire 4 in the direction of which the guide wire 4, which is fixed in place at two fixation points, attempts to expand on the basis of its inherent elasticity. In the present case, these two fixation points of the guide wire exist on its progression path between the contact points or surfaces at the exit location of the passage opening 12 from the cover plate 13 of the attachment part 2 and at the deflection and clamping part 9. On this progression path, the inherently elastic guide wire 4 attempts to expand in a direction essentially perpendicular to this progression path, with the consequence that it presses against the underside of the deflection and clamping bracket 9 or its bracket parts 9a, 9b, which underside faces toward the guide wire support 3, on its expansion path.

At this point, it should still be noted that the transverse dimension d in question is determined by the diameter of a guide wire 4 having a round cross-section. If, instead, the guide wire used possesses a rectangular cross-section, the transverse dimension in question should be understood, in contrast, to be the one edge length of the one guide wire 4 having a rectangular cross-section, specifically preferably its shorter cross-section edge length in the case of different cross-section edge lengths. In the case of an oval cross-section having a large diameter and a small diameter, in comparison, the transverse dimension of the guide wire is to be understood to be its small diameter.

In FIGS. 1, 2, and 5, the two bracket parts 9a, 9b are formed, in each instance, by a support member 11a or 11b that projects away from the initial region 8 of the guide wire support 3, and by a spherical upper part carried by this member, having a greater cross-section or body volume that projects relative to the cross-section of the related support part 11a or 11b. Here, the two spherical upper parts are also indicated with 9a, 9b, for the same of simplicity, in other words with the same reference symbols that, are used for the two bracket parts—because these spherical upper parts fulfill the actual function of the deflection and clamping bracket 9. Here, the support parts 11a, lib are preferably set back from edges of the slot 10 of the upper parts 9a, 9b carried by them, with their one sides that lie opposite one another, as is clearly evident from the representations according to FIGS. 2 and 5. Here, this slot 10 is situated in the region of the bracket that lies farthest away from the support arms 3a and 3b, and thereby in the center position of the deflection and clamping bracket 9, with reference to the bracket parts or upper parts 9a and 9b. However, the support parts 11a,11b in question can also be configured to be relatively short, in deviation from the conditions presented, and can actually be eliminated entirely, if necessary. In this latter mentioned case, the spherical upper parts 9a, 9b are then attached directly to the guide wire support 3, preferably with a connection region that is almost point-shaped, in any case having a very small contact surface as compared with the cross-section of the upper part.

Here, the spherical upper parts 9a, 9b of the two bracket parts are provided at a distance from one another, forming the said slot 10. In this regard, this slot 10 is preferably formed by flattened surfaces in the sides of the spherical upper parts 9a and 9b that lie opposite one another. In this regard, the slot 10 preferably has a straight-line progression, as is clearly evident from FIG. 5, which shows a detail of the guide wire holder 1 shown in FIG. 1, in the viewing direction A indicated with an arrow there. Furthermore, FIG. 5 shows that the support parts 11a,11b are set back with their outsides, relative to the outsides of the upper parts 9a and 9b carried by them, which project in cross-section. As a result, the guide wire 4 can make contact under the spherical regions of the upper parts 9a and 9b, which are not directly connected with the support parts 11a,11b. The support parts 11a, 11b in question can furthermore be connected with the upper parts 9a and 9b also at such location, so that they end flush with the sides of the upper parts 9a and 9b that form the slot 10, with their sides that lie opposite one another. In any case, the guide wire 4 can be held securely under the two spherical upper parts 9a and 9b in this way, as is also evident from FIGS. 1 and 2.

In general, the slot 10 can also have a different progression shape than the one shown, in other words an arc-shaped progression, for example. Here, the slot 10 runs at an optimal angle α of about 45° with reference to the progression path or accommodation path and holding in place path of the guide wire 4 on the guide wire support 3, in the case of the structure of the deflection and clamping bracket 9 that is evident from FIGS. 1 and 2; here, this means with reference to the longitudinal direction of the guide wire 4, as is also evident from FIG. 5, in general, here the angle a can amount to between 5° and 175° with reference to the accommodation path and holding in place path of the guide wire 4 on the guide wire support 3, or with reference to the longitudinal direction of the guide wire 4 in the region in which the guide wire 4 is able to lie against the inside of the deflection and clamping bracket 9, due to its inherent elasticity.

The medical guide wire 4, which possesses inherent elasticity, is first deflected, for its accommodation and for its holding in place in the guide wire holder 1, at an upper edge of a passage opening 12 of a cover plate 13 of the attachment part 2, at a distance from the attachment part 2, in a direction that leads away from its passage direction in the attachment part 2, and, when this happens, as is evident from FIGS. 1 and 2, is held clamped under the spherical upper parts 9a and 9b that belong to the two bracket parts, before it reaches the termination location 5 between the two support arms 3a and 3b, which represent an additional or further guide wire deflection element, at which location a friction effect is exerted on the guide wire 4. This termination location 5 can be formed by an additional guide wire deflection element 14, which is formed by a guide wire contact surface on the top of the guide wire support 3. On this guide wire contact surface, the guide wire 4 can be guided along a guide path, by means of which a friction resistance can be exerted in the region of the guide wire contact surface in question, relative to the guide wire that is passed over or on the top of the guide wire support 3.

Here, the additional guide wire deflection element 14 is formed by a simple arc surface, directed toward the attachment part 2, according to FIGS. 1 and 2, which surface, like the accommodation path and holding in place path of the guide wire 4, can have a material such as rubber or silicone, which exerts a friction resistance on the guide wire 4 that makes contact with it.

In deviation from the structure of the additional guide wire deflection element 14 explained above, this element can be formed by means of a roller device contained in the guide wire support 3, in the form of a single roller member. This roller member can be a fixed roller member or a roller member that can only be turned with difficulty when the guide wire 4 is pulled over this roller member. In both cases, the respective roller member can possess a rough surface, so that a friction resistance is exerted counter to the guide wire 4, which is pulled over this roller member.

Alternatively to this, the aforementioned roller device can be formed by two roller device parts that are brought, together with axial or radial resilience, between which parts the guide wire 4 can be accommodated. In this case, too, the two roller device parts can each possess a rough surface, so that here, too, a friction resistance is exerted counter to the guide wire 4 that is pulled over this roller member.

The aforementioned roughness of the guide wire contact surface 9 can be achieved, for example, by means of structuring of this surface and/or by means of a correspondingly rough surface covering that is applied to the surface in question.

To work with the guide wire 4 in connection with a medical device (not shown), such as an endoscope, the guide wire 4 is first introduced through the opening 12 of the cover plate 13 of the attachment part 2 set onto the medical device in question, to such an extent until the tip (not shown) of the guide wire 4 has been advanced into a desired target region.

Then, the section of the guide wire 4 that projects out of the opening 12 of the cover plate 13 of the attachment part 2 is laid into the guide path on the top of the guide wire support 3, through the slot 10 that is present there, after it has been introduced into the guide wire deflection member 9, so that it comes into contact with the said one termination location 5 and the additional deflection element 14. Subsequently, the part of the guide wire 4 that has been conducted downward around this deflection element 14 and lies the farthest away from the attachment part 2 is laid around the offset region 7 of the guide wire support 3, which region represents yet another guide wire deflection element, specifically around one of the support arms 3a and 3b of this guide wire support 3—here, around the support arm 3a. As a result, the guide wire 4 is deflected from its guide path, on the top of the guide wire support 3 at an angle, specifically by about 90°, as is evident from FIG. 2. As a result, the guide wire 4 is more or less “tidied up” in a desired direction, in which direction it can now no longer be displaced on the guide wire support 3 in its longitudinal direction.

The contact points of the guide wire 4 at the exit location from the cover plate 13 of the attachment part 2, at the guide wire deflection member 6 on the top of the guide wire support 3 and at the deflection element 14 on the top of the guide wire support 3, above all, are decisive for this non-displaceability of the guide wire 4 in its longitudinal direction on the guide wire support 3. The contact points of the guide wire 4 on the offset region 7 of the guide wire support 3 has a supporting effect on the non-displaceability of the guide wire 4 in its longitudinal direction on the guide wire support 3, as explained above.

The two bracket parts 9a, 9b of the guide wire deflection member 14 can now also be structured differently than explained above using FIGS. 1, 2, and 5. Thus, for example, one of the two bracket parts 9a, 9b can have a support member that projects away from the initial region of the guide wire support 3, such as the support member 11a or 11b, and an upper part 9a or 9b carried by this member, having a cross-section or body volume that projects relative to the cross-section of the support part in question, and the other bracket part, in other words 9b or 9a, can merely be a part adjacent, to the said upper part, which is provided with one side at a distance of the slot width from the upper part 9b or 9a in question, of the one of the two bracket parts.

In this case, the slot 10, with its slot width w that corresponds to the transverse dimension d of the guide wire 4, can be provided only in the region of the projecting cross-section or body volume of the upper part of the one bracket part 9a or 9b and the support part that forms the other bracket part 9b or 9a, and, if necessary, the support member that carries this upper part, is set back with its side that faces toward the slot, relative to the latter.

Another configuration possibility for the two bracket parts 9a, 9b of the guide wire deflection member 9 can be seen in FIGS. 3 and 4. Here, the two bracket parts 9a′, 9b′ are formed by arc-shaped bracket parts having a round cross-section, for example, which parts are separated from one another by means of a slot 10′ in the region of their locations that project farthest away from the guide wire support 3, for the dimensioning of which slot the same holds true as was explained above with regard to the slot 10. The slot 10′, like the slot 10, runs at a slant (preferably at an angle of about 45°) relative to the longitudinal direction of the guide wire 4 accommodated by the guide wire holder 1. However, the slot 10′ could also run parallel to the longitudinal direction of the guide wire 4 accommodated by the guide wire holder 1, if it were present in a lateral region of the bracket that comprises the two bracket parts 9a′ and 9b′.

In the case that the two bracket parts 9a, 9b or 9a′, 9b′ have such an elasticity that they can be pressed apart from one another, such as when the guide wire 4 is pressed through the slot formed between them, the aforementioned slot, which runs parallel to the longitudinal direction of the guide wire 4 accommodated by the guide wire holder 1, could also be formed at the locations situated farthest away from the guide wire support 3. In this case, the slot in question could have a slot width that lies below the aforementioned transverse dimension of the guide wire 4.

Yet two further design possibilities for the two bracket parts 9a, 9b of the guide wire deflection member 9 are shown in FIGS. 6 and 7.

According to FIG. 6, the respective upper part—here indicated with 9a″ or 9b″—is configured in the shape of a roller, roll or barrel with its cross-section or body volume that projects relative to the cross-section of its support part, not indicated in any further detail. A slot is present between these upper parts 9a″ or 9b″ that are roller-shaped, roll-shaped or barrel-shaped, just like between the bracket parts 9a and 9b of the exemplary embodiments explained above.

According to FIG. 7, the respective upper part—here indicated with 9a″′ or 9b″′—is configured in the shape of a cone with its cross-section or body volume that projects relative to the cross-section of its support part, not indicated in any further detail. Here, “cone-shaped” is understood to mean both a full cone shape and a truncated cone shape. A slot is present also between these cone-shaped upper parts 9a″′ or 9b″′, just like between the bracket parts 9a and 9b of the exemplary embodiments explained above.

In the case of the embodiments of the upper parts 9a″, 9b″ according to FIG. 5 and 9a′″, 9b′″ according to FIG. 7, their support parts can be structured in pin shape or cylinder shape, in each instance. However, the support parts in question can, as an alternative to this, also be formed by crosspiece parts, with which the upper parts, of the respective upper part pair are connected with the guide wire support 3, proceeding from their ends that lie opposite one another. These crosspiece parts preferably extend at most up to ox to shortly before the longitudinal center of the respective upper part.

In conclusion, it should still be noted that the attachment part 2 can consist of a biocompatible material, such as a metal or plastic, for example of a rubber material. All the other elements of the guide wire holder 1 described here also consist of a biocompatible material, such as of a metal or plastic, such as, for example, of an ABS plastic (acryl nitrile butadiene styrene copolymerizate), specifically preferably as a single, cohesive molded part or injection-molded part. In this regard, edges of parts of the guide wire, holder 1, along which the guide wire 4 is guided, are preferably rounded off, in order to prevent damage to the guide wire surface and to simplify insertion of the guide wire 4 into the guide wire holder 1 for the user.

The guide wire 4, which demonstrates inherent elasticity, consists, as known, of a stainless steel core that has been sheathed by means of polytetrafluoroethylene—PTFE (trademark: Teflon).

### REFERENCE SYMBOL LIST

1 guide wire holder
2 attachment part
3 guide wire support
4 guide wire
5 termination location
6 connection part
7 offset region
8 initial region
9 deflection and clamping bracket
9a, 9bbracket part, upper part
9a′, 9b′ bracket part, upper part
9a″, 9b″ bracket part, upper part
9a″′, 9b″′ bracket part, upper part
10, 10′slot
11a,11b support member
12 passage opening
13 cover plate
14 guide wire deflection element, arc surface
A viewing direction
α angle
d transverse dimension/diameter of the guide wire 4
w slot width

1. Guide wire holder (1) for accommodation and holding in place of a medical guide wire (4) that possesses inherent elasticity, and for attachment to a medical device, particularly to an endoscope, having an attachment part (2) through which the guide wire (4) can be passed and a guide wire support (3) connected with the attachment part (2) and projecting away from the latter, which support permits the guide wire (4) to be held in place by means of a guide wire accommodation device (9, 5, 7), which device contains a first guide wire deflection member (9) that deflects and clamps the guide wire (4) at a distance from the attachment part (2), in a direction leading away from its passage direction in the attachment part (2), which member has a deflection and clamping bracket (9) that is open on one side and rises from a flat initial region, which bracket allows the guide wire (4) to be accommodated in an opening and released from the latter again, and is followed, at further distances from the attachment part (2), by an additional guide wire deflection element (5) and yet a further guide wire deflection element (7), wherein the deflection and clamping bracket (9) has two bracket parts (9a, 9b), which project, away from the initial region of the guide wire support (3) and between which a slot that forms the said opening is provided, which slot has a slot width that lies below the transverse dimension of the guide wire in the case of bracket parts that can be pressed elastically apart from one another, and a slot width that corresponds to the transverse dimension of the guide wire (4) in the case of rigid bracket parts, and which is situated between the two bracket parts (9a, 9b) in a position and/or alignment outside of a guide wire expansion path of the guide wire support (3), which path is determined by the inherent elasticity of the guide wire (4).

2. Guide wire holder (1) according to claim 1, wherein one of the two bracket parts (9a, 9b) has a support member (11a; 11b) that projects away from the initial region (8) of the guide wire support (3), and an upper part (9a; 9b) carried by this member, having a cross-section or body volume that projects away relative to the cross-section of the support part (11a, 11b), and wherein the other bracket part (9b; 9a) is merely a support part (11b) adjacent to the said upper part (9a; 9b), which support part is provided with one side at a distance from the respective upper part (9a; 9b) of the one of the two bracket parts (9a; 9b), forming the slot (10).

3. Guide wire holder (1) according to claim 2, wherein the slot (10) is provided only in the region of the projecting cross-section or body volume of the upper part (9a; 9b) of the one bracket part (9a; 9b) and the support part (11b; 11a) that forms the other bracket part (9b; 9a), and wherein the support member (11a; lib) that carries this upper part (9a; 9b) is set back, if necessary, with its side that faces toward the slot (10), opposite the latter.

4. Guide wire holder (1) according to claim 1, wherein each of the two bracket parts (9a, 9b) has a support member (11a; 11b) that projects away from the initial region (8) of the guide wire support (3), and an upper part (9a; 9b) supported by the latter, having a cross-section or body volume that projects relative to the cross-section of the related support part (11a; 11b), and wherein the upper parts (9a; 9b) of the two bracket parts are provided at a distance from one another, forming the said slot (10).

5. Guide wire holder (1) according to claim 4, wherein the support parts (11a; 11b) are set back, if necessary, from edges of the slot (10) of the upper parts (9a, 9b) supported by them, with sides that lie opposite one another.

6. Guide wire holder (1) according to claim 2, wherein the respective upper part (9a, 9b), with its cross-section or body volume that projects relative to the cross-section of its support part (11a; 11b), is configured in the shape of a roller, roll, cone, barrel or sphere.

7. Guide wire holder (1) according to claim 2, wherein the respective upper part (9a, 9b) forms a flattened surface toward the slot (10).

8. Guide wire holder (1) according to claim 1 wherein the slot (10) has a straight-line progression.

9. Guide wire holder (1) according to claim 1, wherein the slot (10) provided in the central region of the deflection and clamping bracket (9), between its two bracket parts (9a, 9b), runs at an angle a between 5° and 175° with reference to the accommodation and holding in place path of the guide wire (4) on the guide wire support (3).

10. Guide wire holder (1) according to claim 1, wherein the guide wire support (3) has a material such as rubber or silicone in the region of the accommodation and holding in place path of the guide wire (4), which material exerts a friction resistance on the guide wire (4) that lies against it.
